(19) United States
(12) Patent Application Publication
(10) Pub. No.: US 2026/0165589 A1
(43) Pub. Date: Jun. 18, 2026

(54) NON-INVASIVE SENSOR FUSION AND A1 TECHNOLOGY FOR HUMAN VITAL MEASUREMENT

(71) Applicant: BLUESEMI RESEARCH & DEVELOPMENT PRIVATE LIMITEtD, Gachibowli, Hyderabad, Telangana (IN)

(72) Inventor: SUNIL KUMAR MADDIKATLA, Andhra Pradesh (IN)

(73) Assignee: BLUESEMI RESEARCH & DEVELOPMENT PRIVATED LIMITED, Gachibowli, Hyderabad, Telangana (IN)

(21) Appl. No.: 18/674,535

(22) PCT Filed: Nov. 26, 2022
(86) PCT No.: PCT/IN2022/051031

§ 371 Date: May 24, 2024

(30) Foreign Application Priority Data
Nov. 26, 2021 (IN) 202141054751

(51) Int. Cl. A61B 5/0205; A61B 5/00; A61B 5/021; A61B 5/024; A61B 5/145; A61B 5/1455; A61B 5/346

(52) U.S. Cl. CPC A61B 5/02055; A61B 5/0002; A61B 5/7207; A61B 5/725; A61B 5/7267; A61B 5/021; A61B 5/02433; A61B 5/14532; A61B 5/14552; A61B 5/346

(57) ABSTRACT

The present disclosure relates to a non-invasive sensing system for measuring key body vitals. The system includes a sensing device to generate a pre-processed signal in response to the lights reflected by the fingertips of a user. The pre-processed signal is received by a client application for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. The system also includes a user interface, of the client application, to receive a request for the measured key body vitals and to output the measured key body vitals.

### FIELD OF THE INVENTION

The present disclosure relates generally to non-invasive sensor fusion and artificial intelligence (AI) technology. In particular, the present subject matter relates to a non-invasive sensing system for measuring key body vitals and a method for operating the same.

### BACKGROUND

Leading a healthy lifestyle is becoming a concern of great tribulation in recent times. A key to leading a healthy lifestyle is to know how your body has been performing and have an analysis of the very simple things that can have colossal effects on your vitals. Understanding basic vitals such as heart rate, oxygen saturation (Spo2), blood glucose, blood pressure, heart rate variability (HRV), etc, can facilitate one to lead a healthy lifestyle.

However, current methods of measuring these key vitals, for example, blood sugar level are agonizing due to the invasive procedures they follow to measure the blood sugar level. No matter how small and thin the needle is, the pain is still there. While some people get used to it and hence bear it, it can be problematic for others. Measuring blood glucose levels is an everyday thing for diabetics, so it becomes difficult to incorporate this pain into your routine In 2014, 8.5 percent of adults aged 18 years and older had diabetes. In 2019, diabetes was the direct cause of 1.5 million deaths and 48 of all deaths due to diabetes occurred before the age of 70 years. Another 460000 kidney disease deaths were caused by diabetes, and raised blood glucose causes around 20 percent of cardiovascular deaths”—The World Health Organisation (WHO).

The above information from WHO interprets how significantly catastrophic diabetes can be. A small step towards avoiding this catastrophic effect on the user can be a regular measurement of the vitals and an understanding of blood glucose levels daily for lifestyle improvements.

A Product that can measure the key vitals above discussed in a way where there are no agonizing invasive procedures can bring compelling results and help us to adapt to lead a healthy life.

Therefore, there is a need in the art for a system and method to provide non-invasive procedures to measure the key body vitals and help users to lead healthy life

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key nor critical elements of all embodiments, nor delineate the scope of any or all embodiments.

The present disclosure, in one or more embodiments, relates to a non-invasive sensing system for measuring key body vitals and a method for operating the same. In an aspect, the proposed system includes a sensing device and a computing coupled to the sensing device.

In one embodiment herein, the sensing device includes a light emitting unit, a photodetector, an elementary filter, a first in first out (FIFO) data registers, a pre-processing module, and a communication module.

In one embodiment herein, the light-emitting unit is mounted underneath the top glass. The light-emitting unit comprises two sets of light-emitting diodes to emit two different wavelengths into the fingertip of the user when the user places the fingertip on the top glass. The two sets of light-emitting diodes include a Light Emitting Diodes (LED) with a wavelength of 660 nm, a Near Infra-Red LEDs (NIRs) with a wavelength of 880 nm.

In one embodiment herein, the photodetector, mounted underneath the top glass, is configured to receive the light reflected from the fingertip and convert the light into a Photoplethysmography (PPG) signal. In an aspect, the photodetector is configured with spectral range sensitivity of 600 to 5000 nm. Further, the top glass allows wavelength frequencies of 600 nm to 900nm with about 85%-95% transmission percentage. Also, the system includes a black optical sensor shield that covers the boundaries of the light emitting unit and the photodetector for absorbing unwanted light that might bounce back to the photodetector.

The elementary filter is configured to cooperate with the photodetector to receive the PPG signal, filter the PPG signal to remove noise frequencies that are not containing information about the key body vitals, and generate a denoised signal. In an aspect, the elementary filter is a Finite Impulse Response (FIR) band-pass filter with band frequencies from 0.5 Hz to 5.0 Hz and a gain of 1100, wherein the FIR band-pass filter is configured to remove the noise frequencies lying beyond a frequency range of 0.5 Hz to 5.0 Hz.

Further, the FIFO data registers are provided for linear processing of the denoised signal generated by the elementary filter. Then, the pre-processing module is configured to receive the denoised signal from the elementary filter. The denoised signal is obtained as a matrix of signals (n×7), where said matrix of signals includes 3000 data samples with a sampling frequency of 100 samples per second and a sampling period of 30 seconds, and where said pre-processing module is configured to pre-process the denoised signal to generate a pre-processed signal.

In one embodiment herein, the pre-processing module is configured to receive the matrix of signal (n×7) to perform concatenation of two parts consisting in the matrix of signals (n×7) including 3000 data samples to generate a concatenated signal, where the first part of the matrix of signals (n×7) lies in 3rd column of the matrix and a second part of the matrix of signals (n×7) lies 1st column of the matrix.

In one embodiment herein, the pre-processing module is configured for trimming of the concatenated signal to discard a part from the beginning of capturing the PPG signal and a part from the ending of capturing the PPG signal to generate a trimmed signal, where the part from the beginning includes about 300 data samples and the part from the ending includes about 100 data samples from the array of 3000 data samples.

In one embodiment herein, the pre-processing module is configured for performing Ultra-low pass conditioning to the trimmed signal to generate a Ultra-low pass conditioned signal. In one embodiment herein, the pre-processing module is configured for generating the pre-processed signal as a pre-processed difference signal computed based on a difference between the Ultra-low pass conditioned signal and the trimmed signal.

Further, the communication module is coupled to the pre-processing module to transmit the pre-processed signal. The preprocessing signal is received by a client application, and executed by a processor on a computing device, for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. In an aspect, the user interface of the client application is configured to request the measured key body vitals and to output the measured key body vitals. Further, the client application may include a blood glucose module, a blood sugar module (HbA1C), an ECG module, a heart rate module, a blood pressure module, and a body temperature module.

For blood glucose measurement, the blood glucose module is configured to receive the ultra-lowpass conditioned signal from the pre-processing module of the sensing device. The blood glucose module comprises a glucose conditioning module to receive the Ultra-low pass conditioned signal from the pre-processing module of the sensing device and compute Normal value of the key two dimensional components analyzed by AI a conditioning interference module to receive the computed Normal as a parameter, wherein the conditioning module is configured to compute a user's blood glucose value based on a range in which the computed normal value falls, wherein for computing the user's blood glucose value, the conditioning interference module uses a polynomial equation where one of the key inputs is the computed Normal value and coefficients for each range are determined by pre-training a regression classifier of the AI-based model with badging.

For blood sugar measurement (HbA1C), the blood sugar module is configured to receive the pre-processed difference signal from the pre-processing module of the sensing device. The blood sugar module is configured to perform batching the pre-processed difference signal into an M number of batches of an N number of samples, where N=50 for each batch, simultaneously computing a peak-to-peak value and an average peak-to-valley for each peak in the batch, computing median reference values of the peak-to-peak and the peak-to-valley values of entire batches of the key components of overall length of the difference signal analyzed by the AI categorization, and implementing the pre-trained AI-based model to receive the median reference values as the extracted features for predicting blood sugar (HBA1C).

For ECG measurement, the ECG module is configured for receiving the denoised signal as the matrix of signal (n×7). The ECG module is configured to perform motion artifact removal from the denoised signal. The ECG module is configured for processing the motion artifact removed signal to identify the heart rate with multiple frequency filters, wherein the heart rate is present only in the frequency range of 0.663 to 3.66 Hz, and passing the filtered signal through a series of thresholding functions, wherein each peak that is observed after the set threshold functions is considered as a heart beat, and a number of peaks which are detected in the signal for a defined time frame with respective their timestamp are utilized to generate an ECG report to understand sinus rhythm of the heart.

The hardware unit also comprises of a set of Dry electrodes which acts as one of the sensors for ECG module. For heart rate measurement, the client application further comprises a heart rate module to perform receiving the denoised signal as the matrix of signal (n×7), perform motion artifact removal from the denoised signal, processing the motion artifact removed signal to identify the heart rate with multiple frequency filters, wherein the heart rate is present only in the frequency range of 0.663 to 3.66 Hz, passing the filtered signal through a series of thresholding functions, wherein each peak that is observed after the set threshold functions is considered as a heart beat, and a number of peaks detected in the signal for a defined time frame with respect to their timestamp are utilized to measure the heart rate of the user.

For blood oxygen level (SPo2) measurement, the blood oxygen level (SPo2) module is configured to perform receiving the PPG signal at the photodetector, measuring saturation of blood by oxygen from PPG signal received at two wavelengths including red signals received from LED and IR signals received from NIR, calculating the blood oxygen (SpO2) level is calculated from a calibration R curve plotting SpO2 versus ratio red and IR signals, wherein the R is calculated as R=(Rac/Rdc)/(IRac/IRdc), where Rac is the pulsating AC component of the fingertip by red signal and Rdc is the non-pulsating DC component of the fingertip by a red signal of wavelength 660 nm, and IRac and IRdc are also pulsating AA and DC components of the fingertip by the IR signal of wavelength 880 nm.

For blood pressure measurement, the blood pressure module is configured to perform separating the denoised signal obtained as a matrix of signals (n×7) into two component signals: red component and irr component, wherein the red component is formed by concatenating zero-indexed columns 3 and 1, which denotes the signal that is obtained from the reflected light of red signal of wavelength 660 nm, and wherein the irr component is formed by concatenating the zero-indexed columns 2, 4 which denotes the signal that is obtained from the reflected IR light of wavelength 880 nm, Ultra Lowpass conditioning and trimming the signal. The Ultra Lowpass conditioned signal and trimmed components (r curr, i curr) by the normal of the respective component signals for the red component (r curr), normalising it and computing its inverse representation (r curr inv) by multiplying 1.01 by a maximum value in this normalised signal and then subtracting each element of the normalised signal from this value; for the irr component (i curr), multiply 1.01 by a maximum value in the irr component and then subtract each element of the signal from this value to obtain i curr inv, finding indices of peaks in the signals i curr inv and i curr as maxindices and minIndices, implementing the maxindices to index into r curr inv to get an array of values (r curr inv maxima), implementing minIndices to index into r curr inv to get another array of values (r curr inv minima), converting both of these arrays to the same length by only considering values up until the length of the smaller of the two arrays; and once both of these arrays are obtained as two arrays of equal length, computing their respective normals, add both their normals up and divide this sum by 2 to obtain the input to the equations for computing the systolic and diastolic blood pressure values, wherein the equations for both systolic and diastolic blood pressure are Polynomial equations, whose coefficients are determined by a pre-training a regression classifier of the AI-based model.

For measurement of the body temperature, the temperature sensor is configured to measure the temperature of the user based on the IR signal of wavelength 880 nm obtained from the NIRs.

In one embodiment herein, for operating a non-invasive sensing system for measuring key body vitals. The method includes receiving, by a top glass of a sensing device, a touch of a fingertip of a user. The method also includes emitting, by a light emitting unit mounted underneath the top glass with two sets of light emitting diodes, two different wavelengths into the fingertip of the user when the user places the fingertip on the top glass. The method also includes receiving, by a photodetector mounted underneath the top glass, the light reflected from the fingertip to convert the reflected light into a Photoplethysmography (PPG) signal. The method also includes receiving the PPG signal by an elementary filter configured to cooperate with the photodetector. The method also includes filtering, by the elementary filter, the PPG signal to remove noise frequencies that are not containing information about the key body vitals for generating a denoised signal.

The method also includes receiving, by a pre-processing module, the denoised signal from the elementary filter, wherein the denoised signal is obtained as a matrix of signals (n×7), wherein said matrix of signals includes 3000 data samples with a sampling frequency of 100 samples per second and a sampling period of 30 seconds. The method also includes pre-processing, by the pre-processing module, the denoised signal to generate a pre-processed signal. The method also includes transmitting the pre-processed signal by a communication module coupled to the pre-processing module. The method also includes receiving, by a client application executed by a processor on a computing device, the pre-processed signal for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. The method also includes outputting, by a user interface of the client application, the measured key body vitals in response to a request received from the user.

Further, as per another embodiment of the present invention, Differential Analysis of blood can be done by using more than one finger of the user, at a time, at the time of data collection. The analysis of blood by more than one finger of the user, at the time of data collection, forms a closed loop around the body and understands differential vital parameters more accurately. The Entire computation modules described above for the accurately measurement of key body vitals including blood glucose, blood sugar (HBA1C), electrocardiogram (ECG), heart rate, blood oxygen level (SPo2), blood pressure, and body temperature are computed from the signals collected from, for example, both the left and right fingers of the user, at a time, at the time of data collection. The vital results have shown better correlation if the system can understand the minor variations in both the fingers there by producing unified results.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a high-level network architecture of a non-invasive sensing system for measuring key body vitals, in accordance with embodiments of the invention.

FIG. 2 illustrates an exemplary block diagram of the sensing device, in accordance with embodiments of the invention.

FIG. 4A-4C illustrate different exemplary views of the sensing device, in accordance with embodiments of the invention.

FIG. 5 illustrates an experimental outcome of the implementation of the lights with different wavelength of the sensing device, in accordance with embodiments of the invention.

FIG. 6 illustrates a functional flowchart of a pre-processing module of the sensing device, in accordance with embodiments of the invention.

FIG. 7 illustrates an exemplary functional block diagram of a computing device coupled to the sensing device, in accordance with embodiments of the invention.

FIG. 8 illustrates an exemplary functional block diagram of a blood glucose module coupled to the sensing device, in accordance with embodiments of the invention.

FIG. 9 illustrates an exemplary functional block diagram of a blood sugar module coupled to the sensing device, in accordance with embodiments of the invention.

FIG. 10 illustrates an exemplary functional block diagram of a heart rate module coupled to the sensing device, in accordance with embodiments of the invention.

FIG. 11 illustrates an exemplary functional block diagram of a blood pressure module coupled to the sensing device, in accordance with embodiments of the invention.

FIGS. 12A-12B illustrate an exemplary flow diagram illustrating a method of operating a non-invasive sensing system for measuring key body vitals, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

Embodiments of the present disclosure relate to a key tool to understand human key body vitals in a way where there are no agonizing invasive procedures can be spectroscopy. Spectroscopy allows the user to understand human key body vitals with great detail and minimum user discomfort. Spectroscopy allows us to measure Photoplethesmography also known as PPG which has been playing a significant role in the measurement of heart rate, oxygen saturation (Spo2), etc. Having this backdrop, the present disclosure proposes an in-depth multilevel analysis of a PPG signal with combinations of various components of the signal combined with different wavelengths at which the signal is captured giving a greater understanding of human key body vitals and measurement of key body vitals, such as blood glucose, non-invasively.

As per the inventive implementation of the present disclosure, the measurement of the key body vitals is initiated with a user placing the tips of his/her thumb on a sensing device (hardware device). Then, sensors embedded in the device, consisting of Light Emitting Diodes (LEDs) with a wavelength of 660 nm and a Near Infra-Red LED (NIR) with a wavelength of 880 nm, emit light on the tip of the thumb of the user, and a photodetector with spectral range sensitivity of 600 nm to 5000 nm receives reflected light from the tip of the thumb of the user. In the present disclosure, a particular fusion of LEDs with 2 different wavelengths has been chosen for an optimal understanding of blood to accurately measure the key body vitals.

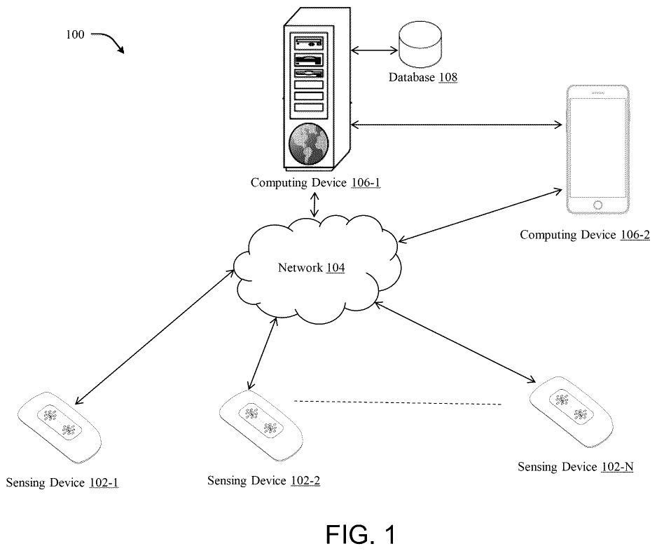

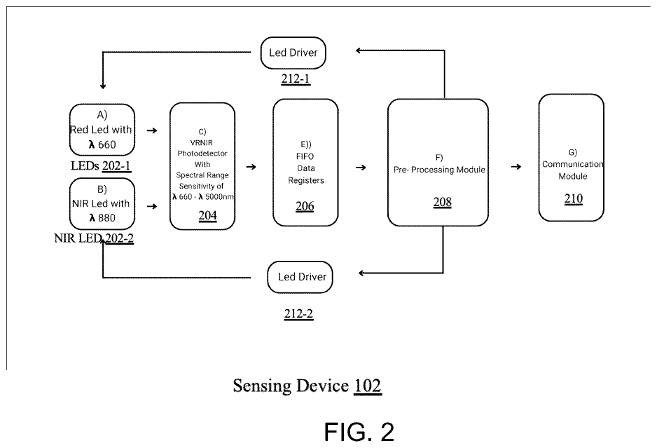

FIG. 1 refers to an exemplary architecture for implementing a non-invasive sensing system 100 for measuring key body vitals in accordance with an exemplary embodiment of the present disclosure. In an aspect, the system 100 of the present disclosure can include a plurality of sensing devices 102-1, 102-, 102-N, hereinafter collectively referred to as sensing devices 102 and individually as sensing device 102. In an aspect, the sensing device 102 is a hardware unit consisting of mainly five different blocks as shown in FIG. 2. The sensing device 102 comprises a top glass configured to receive a touch of a fingertip of a user and generate a pre-processed signal. The sensing device 102 may communicate with other sensing devices 102 over a communication network 104. In an aspect, the communication network 104 can be 3G, 4G, 5G, 6G, or any suitable wireless communication network.

The sensing device 102 may be in communication with a computing device 104-1, 104-2 over the communication network 104. Once the physical sensing of body parameters is performed, the pre-processed signal from the sensing device 102 is transmitted to a client application executed by a processor on the computing device 106, to receive the pre-processed signal for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. Further, the client application includes a user interface to receive a request for the measured key body vitals, output the measured key body vitals, and store them in a database 108 coupled to the computing device 106-1, 106-2 for future analysis.

FIG. 2 refers to block diagram of the sensing device 102 in accordance with an embodiment of the present disclosure. In an aspect, the sensing device 102 is a hardware unit consisting of mainly five different blocks as shown in FIG. 2. The sensing device 102 comprises a top glass configured to receive a touch of a fingertip of a user. The top glass allows wavelength frequencies of 600 nm to 900 nm with about 85%-95% transmission percentage. Further, the sensing device 102 includes a light emitting unit mounted underneath the top glass, said light emitting unit comprising two sets of light emitting diodes 202-1, 202-2 to emit two different wavelengths into the fingertip of the user when the user places the fingertip on the top glass; a photodetector 204, mounted underneath the top glass, to receive the light reflected from the fingertip and convert the light into a Photoplethysmography (PPG) signal; an elementary filter 206 configured to cooperate with the photodetector 204 to receive the PPG signal, filter the PPG signal to remove noise frequencies that are not containing information about the key body vitals, and generate a denoised signal; a pre-processing module 208 to receive the denoised signal from the elementary filter 206, where the denoised signal is obtained as a matrix of signals (n×7), where said matrix of signals includes 3000 data samples with a sampling frequency of 100 samples per second and a sampling period of 30 seconds, and where said pre-processing module 208 is configured to pre-process the denoised signal to generate a pre-processed signal; and a communication module 210 coupled to the pre-processing module 208 to transmit the pre-processed signal.

In an aspect, said two sets of light-emitting diodes include Light Emitting Diodes (LEDs) 202-1 with a wavelength of 660 nm; and Near Infra-Red LEDs (NIRs) 202-2 with a wavelength of 880 nm. In an aspect, the sensing device 100 includes a black optical sensor shield that covers the boundaries of the light emitting unit and the photodetector for absorbing unwanted light that might bounce back to the photodetector. Also, the photodetector 204 is configured with spectral range sensitivity of 600 nm to 5000 nm. Further, as can be seen from FIG. 2, the sensing device 102 may include LED drivers 212-1, and 212-2 for controlling the illumination of the LEDs 202-1 and NIRs 202-2. In an aspect, the elementary filter 206 is a Finite Impulse Response (FIR) band-pass filter with band frequencies from 0.5 Hz to 5 Hz and a gain of 1100. The FIR band pass filter is configured to remove the noise frequencies lying beyond a frequency range of 0.5 Hz to 5 Hz. In an alternative aspect, instead of the elementary filter 206 or in addition to the elementary filter 206, the sensing device 102 may include a first in first out (FIFO) data registers for linear processing of the denoised signal generated by the elementary filter.

FIG. 3 refers to perspective view of the sensing device 102 in accordance with an embodiment of the present disclosure.

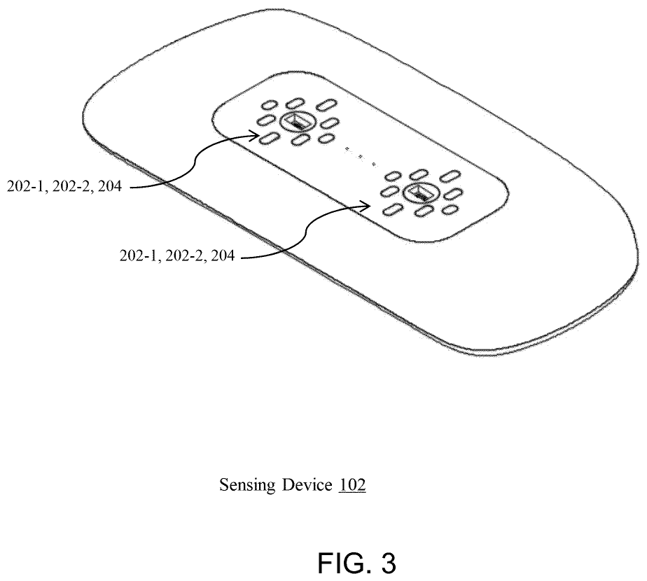

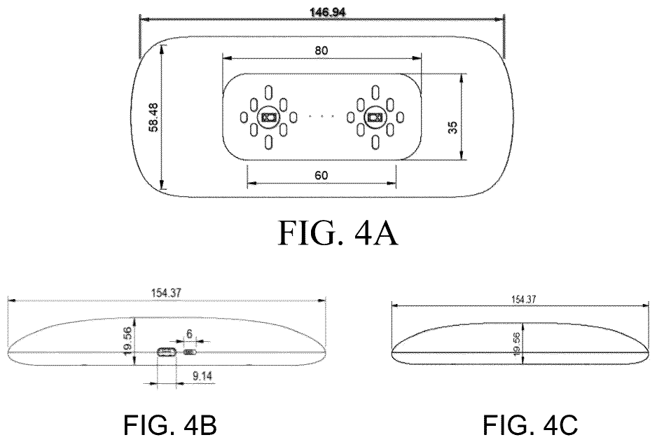

FIGS. 4A-4C refer to different views (front view, top view, and bottom view) of the sensing device 102 in accordance with an embodiment of the present disclosure. The dimensions shown in these figures are for the sake of understanding the exemplary size of the sensing device 102, and are in no way restrictive to the sensing device 102. The shape and size of the sensing device 102 can be varied depending on the required component configuration inside the sensing device 102.

As per the inventive implementation of the present disclosure, the measurement of the key body vitals is initiated with a user placing the tips of his/her thumb on a sensing device (hardware device). Then, sensors embedded in the device, consisting of Light Emitting Diodes (LEDs) with a wavelength of 660 nm and a Near Infra-Red LEDs (NIRs) with a wavelength of 880 nm, emit light on the tip of the finger of the user, and a photodetector with spectral range sensitivity of 600 up to 5000 nm receives reflected light from the tip of the thumb of the user. In the present disclosure, a particular fusion of LEDs with 2 different wavelengths has been chosen for an optimal understanding of blood to accurately measure the key body vitals.

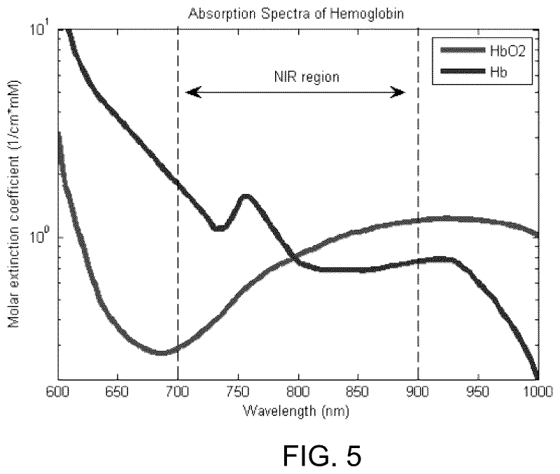

In the present disclosure, a particular fusion of LEDs 202-1 and NIRs 202-2 with two different wavelengths has been chosen for the optimal understanding of blood. An exemplary experimental outcome of the implementation of the lights with different wavelengths is shown in FIG. 5. As shown in FIG. 5, Absorption of light at these wavelengths differs significantly between blood loaded with oxygen and blood lacking oxygen. Oxygenated hemoglobin absorbs more infrared light and allows more red light to pass through. Deoxygenated hemoglobin allows more infrared light to pass through and absorbs more red light. With the understanding of these intrinsic characteristics of blood, further wavelength fusions are under the scope of the present disclosure and can be explored more.

With the sensor (light emitting unit 202-1, 202-2) fusion and photodetector 204 with the right spectral range sensitivity acting as the roots for measuring the PPG signal from the fingertips of the user, here the sensor (light emitting unit 202-1, 202-2) is covered with a hydrolytic resistance class glass (not shown in figures) which acts as a basic cover for the photodetector 204 which compensates for basic motion artifacts and provides a robust base for the measurement of the PPG signal. Once the user places their fingertip on the sensors (light emitting unit 202-1, 202-2), the LEDs and NIRs 202-2 emit the light into the fingertip and the photodetector 204 senses the reflected light, and the photodetector 204 sends the entire data it read to the processing module 208 for further processing which is discussed in detail in the description provided herein below in the present disclosure.

A raw PPG signal consists of a very diverse set of noise contributors to the PPG signal, hence it is very important to understand these noise contributors first, and design/choose filters and methods to remove these noises without compromising the main signal as it sometimes is very easy to eliminate a particular component assuming it to be noise to the signal where it can be a key feature for a vital like glucose. Previous studies show that few particular frequency removal is suggested where components like AC noise/Powerline Interference around the user and minor motion artifacts, etc. are to be understood as noise contributors and needs to be carefully eliminated from the main signal (PPG signal).

The FIR band-pass filter is chosen as the elementary filter 206 with band frequencies from 0.5 Hz to 5 Hz and a gain of 1100, the band frequencies have been chosen where, Components like heart rate, Respiration rate which typically lie in frequencies ranging from 0.75 Hz to 2.0 Hz, etc. are preserved in the main signal for an accurate analysis of vitals like Heart Rate, Blood Pressure, HRV, etc. and only noise from above-mentioned sources like powerline are removed which typically range in 50 Hz. This artifact introduces a sinusoidal component into the recording, at not only its fundamental frequency of 50 Hz, but also as spikes at 100 Hz and its higher harmonics.

After processing the raw PPG signal from the band-pass filter, a signal is left in which noise contributors are present in the same frequency where key body vital information is present. Hence, the further filtering of the filtered PPG signal becomes critically important, and ways in which the noise contributors are found and the delta differences are adjusted for the key body vitals in the PPG signal instead of removing them completely, which may cause feature loss of signal. The denoised signal that is an output of the elementary bandpass filter 206 is transmitted. Different methods such as Single level Discrete Wavelet Transform (DWT) analysis & Multi-level Discrete Wavelet Transform (DWT) were also used to understand the features of the signal in a way where the noise contributors are identified and are compensated in the form of delta correction in the end. The denoised signal is obtained as a matrix of shape (n×7). With a sampling frequency of 100 samples per second and a sampling period of 30 seconds, we will have 3000 samples. This matrix is fed as an input to the algorithms that compute the vital values, which have been described hereinbelow.

The denoised signal is then fed through the pre-processing module 208. The objective of the pre-processing module 208 is to render the denoised signal in a form that is conducive to the analyses and computations for calculating the vitals, especially sensitive key body vitals like glucose needs legitimate signal processing modules like this. The pre-processing module 206 is composed of three steps at first Step concatenation of raw signal columns indexed 3 and 1 (zero-indexed convention). A matrix of signal that is (n×7) consists of the raw signal in two parts, where the first part of the signal lies in the 3rd column of our matrix and the second part of the signal lies in 1st column of only combining both of this in this particular order reveals the original signal, The matrix is designed this way for Data transmission security and signal Protection.

At second Step, Trimming the signal is also a key operation, It is observed that when a user places their fingers on the device a minor movement is observed at the start and end of the sample collection, hence a part of the signal from the beginning (300 values) and a part from the end (100) values are trimmed. A 10 second window allowing users to hold the unit at the beginning of starting the sample collection is also observed as an important operation as this 10 second window allows users to have a firm placement of their fingers on the sensors avoiding any physical movements at the time of sample collection. At third step, an Ultra Lowpass filtering is performed on the trimmed signal and is matched with the length of the original trimmed signal for prevention of data loss.

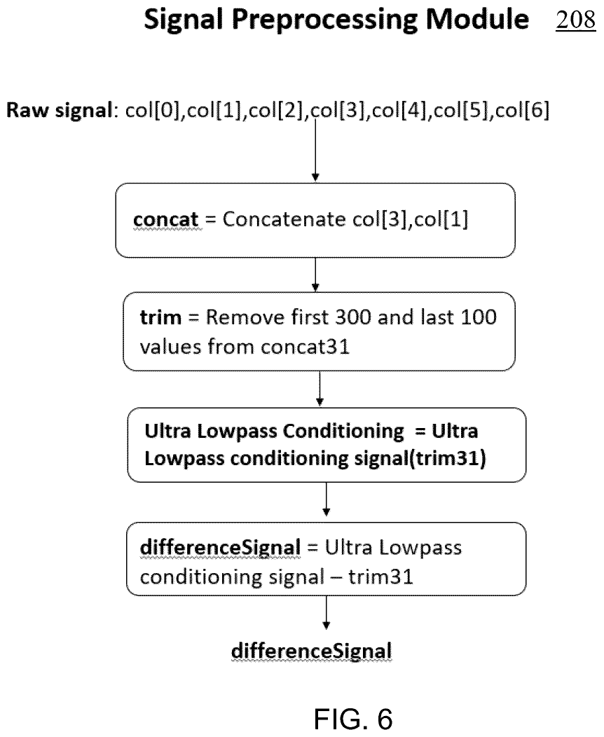

In particular, as shown in FIG. 6, the pre-processing module 208 is configured to receive the matrix of signal (n×7) to perform at step 1: concatenation of two parts consisting in the matrix of signals (n×7) including 3000 data samples to generate a concatenated signal, where a first part of the matrix of signals (n×7) lies in 3rd column of said matrix and a second part of the matrix of signals (n×7) lies 1st column of said matrix. At step 2, trimming the concatenated signal to discard a part from the beginning of capturing the PPG signal and a part from ending of capturing the PPG signal so as to generate a trimmed signal, wherein the part from the beginning includes about 300 data samples and the part from the ending includes about 100 data samples from the array of 3000 data samples. At step 3, performing Ultra Lowpass conditioning on the trimmed signal. At step 4, generating the pre-processed signal as a pre-processed difference signal computed based on a difference between the Ultra Lowpass conditioned Signal and the trimmed signal.

The preprocessing signal from the pre-processing module 208 is received by a client application 708, executed by a processor 702 on a computing device 106, for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. In an aspect, the user interface 706 of the client application is configured to request the measured key body vitals and to output the measured key body vitals.

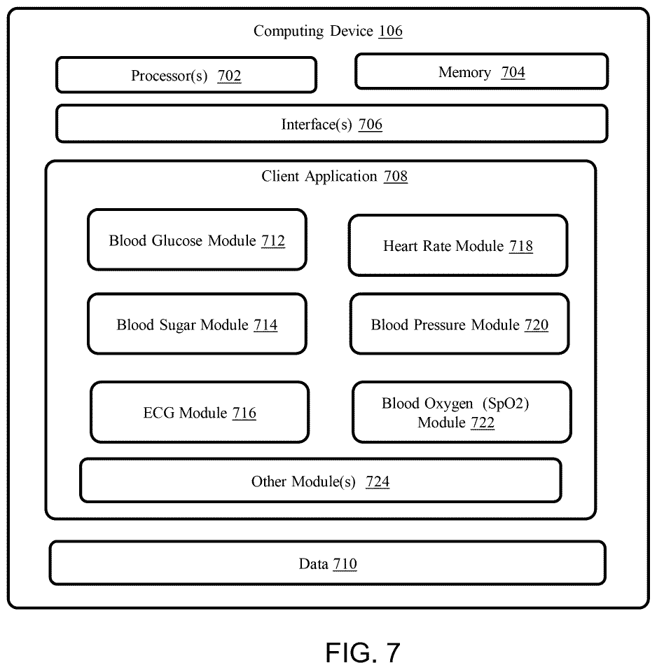

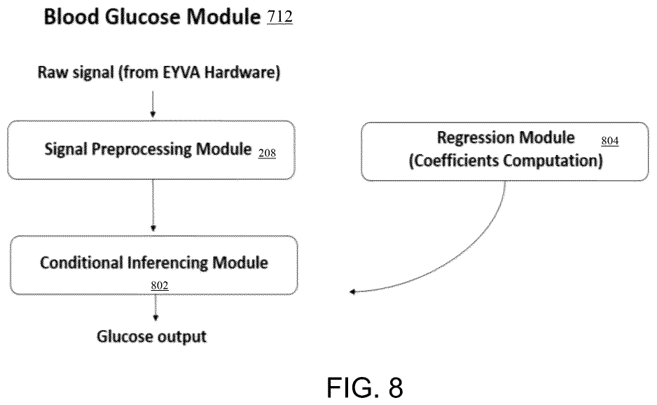

FIG. 7 refers to an exemplary system diagram indicating different functional components of computing device 106, which is coupled to the sensing device 102) in accordance with an exemplary embodiment of the present disclosure. The disclosed computing device 106 for measuring key body vitals can include one or more processor(s) 702. The one or more processor(s) 702 may be implemented as one or more microprocessors, microcomputers, microcontrollers, digital signal processors, central processing units, logic circuitries, and/or any devices that manipulate data based on operational instructions. Among other capabilities, one or more processor(s) 702 are configured to fetch and execute computer-readable instructions stored in memory 704 of the computing device 106. The memory 704 may store one or more computer-readable instructions or routines, which may be fetched and executed to establish end-to-end service between multiple domains. The memory 704 may include any non-transitory storage device including, for example, volatile memory such as RAM, or non-volatile memory such as EPROM, flash memory, and the like.

The computing device 106 may also include an interface(s) 706 (or say, a user interface). The interface(s) 706 may include a variety of interfaces, for example, interfaces for data input and output devices, referred to as I/O devices, storage devices, user interfaces, and the like. The interface(s) 706 may facilitate communication of the computing device 106 with various devices coupled to the computing device 106. The interface(s) 706 may also provide a communication pathway for one or more components of the computing device 106. Examples of such components include, but are not limited to, client application 708 and data 710.

The client application 708 may be implemented as a combination of hardware and programming (for example, programmable instructions) to implement one or more functionalities of the client application 208. In the examples described herein, such combinations of hardware and programming may be implemented in several different ways. For example, the programming for the client application 708 may be processor-executable instructions stored on a non-transitory machine-readable storage medium and the hardware for the client application 708 may include a processing resource (for example, one or more processors), to execute such instructions. In the present examples, the machine-readable storage medium may store instructions that, when executed by the processing resource, implement the client application 708. In such examples, the computing device 106 may include the machine-readable storage medium storing the instructions and the processing resource to execute the instructions, or the machine-readable storage medium may be separate but accessible to computing device 106 and the processing resource. In other examples, the client application 708 may be implemented by electronic circuitry.

The data 710 may include data that is either stored or generated as a result of functionalities implemented by any of the components of the client application 708.

In one embodiment herein, the client application 708 may include a blood glucose module 712, a blood sugar module 714, and an electrocardiogram (ECG) module 716, a heart rate module 718, a blood pressure module 720, a body temperature module 722 and other module 724. The other module(s) 724 may implement functionalities that supplement applications or functions performed by the computing device 106 or the client application 708. The working and operation principles of these modules are described with reference to the following figures.

Blood glucose module 712 is in operation, as shown in FIG. 8, for the blood glucose measurement, the blood glucose module 712 is configured to receive the Ultra Lowpass conditioned Signal from the pre-processing module 208 of the sensing device 102. The blood glucose module 712 comprises a glucose conditioning module to receive the Ultra Lowpass conditioned Signal from the pre-processing module of the sensing device and compute a normal value of the key two dimensional components analyzed by AI, a conditioning interference module 802 to receive the computed Normal value as a parameter, wherein the conditioning module is configured to compute user's blood glucose value based on a range in which the computed normal value falls, wherein for computing the user's blood glucose value, the conditioning interference module 802 uses a polynomial equation where one of the key inputs is the computed Normal value and coefficients for each range are determined by pre-training a regression classifier 804 of the AL based model.

In other words, after processing the PPG signal through the preprocessing module 208 consisting of 3 key operations, a normal is computed on the key two dimensional components analyzed by AI, and this value of the normal is passed as a parameter to the conditional inferencing module 802. In the conditional inferencing module 802, the value of the user's blood glucose is computed based on the range that this normal value falls within. The equation for computing the glucose value is a Polynomial equation where the key input is the normal, value described above and the coefficients for each range are determined by pre-training a regression classifier or a regression module 804. The reason behind choosing to compute the normal on key two dimensional components analyzed by AI rather than the entire signal is because it is observed to give maximum stability and consistent results by restricting the interference of the motion artifacts by the users while adjusting their fingers that might cause variations in the input signals.

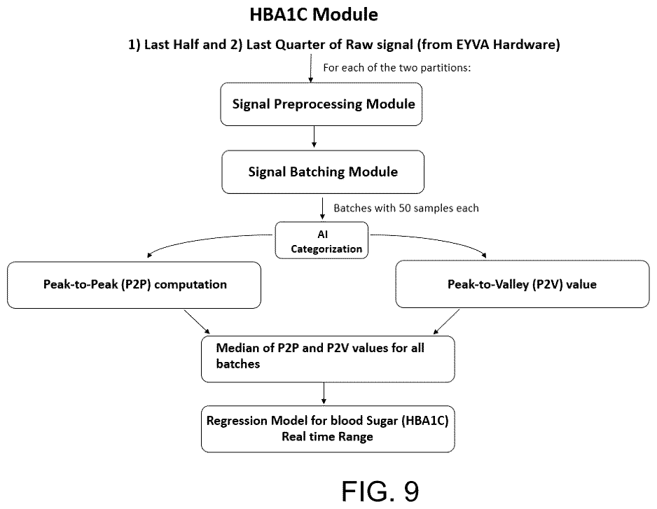

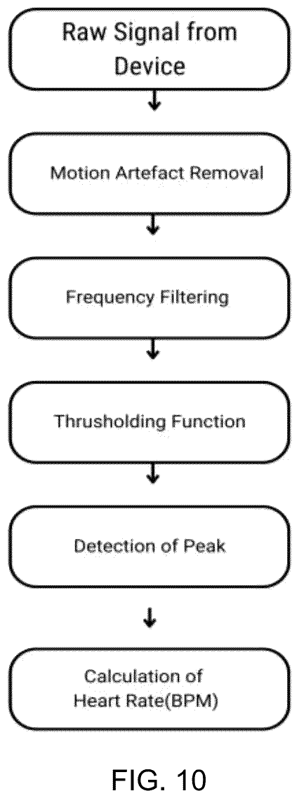

Blood Sugar Module (HbA1c) 714, in operation, as shown in FIG. 9, for blood sugar measurement, the blood sugar module (HbA1c) 714 is configured to receive the pre-processed difference signal from the pre-processing module 208 of the sensing device 102. The blood sugar module is configured to perform batching, by a signal batching module, the pre-processed difference signal into an M number of batches of an N number of samples, where N=50; Computing median reference values of P2P and P2V of the intermediate signal that was generated by the 2 dimensional AI categorization from the preprocessed difference signal. computing median reference values of the peak-to-peak and the peak-to-valley values of entire batches of the key components of overall length of the difference signal analyzed by the AI categorization, implementing the pre-trained AI-based model to receive the median reference values as the extracted features for predicting the blood sugar (HBA1C).

In other words, for HBA1C measurement, the preprocessing steps consist of: the concatenation of raw signal columns indexed 3 and 1 (zero-indexed convention), Ultra lowpass conditioning of the concatenated signal, and computing the difference signal by subtracting the Ultra lowpass conditioned signal signal from the concatenated raw signal. This preprocessed difference signal is then batched into M no. of batches of N samples (where N=50) each. For each batch, we compute the peak-to-peak value and the average peak-to-valley for each peak in the batch. The peak-to-peak value for the batch is calculated by multiplying 2 sqrt(2 ) with the RMS for the signal. On the other hand, for the peak-to-valley computation, the minimum valley that is adjacent to each peak is first found. For most peaks, there will occur a valley before and after the peak value. Then, the minimum valley is determined which will then provide a peak-valley pair. Once this pair is received, the difference is computed between the values of the peak and valley, and then an average difference is computed for each peak-valley pair in the batch. This average value gives the normal peak-to-valley reference. Once the peak-to-peak and normal peak-to-valley values for each batch are computed, the median reference values are computed of the peak-to-peak &-to-valley values of entire batches, batches of the key components of overall length of the difference signal analyzed by the AI categorization. After splitting the signal into M number of batches with N samples in each batch, the median is chosen as a key operation for determining which value to pick from the entire array of computed peak-to-peak & peak-to-valley values. The median eliminates picking any potential motion artifacts in the signal. Motion artifacts usually have higher peak-to-peak & peak-to-valley values hence after arranging the entire array of computed peak-to-peak & peak-to-valley values in ascending order all the motion artifacts tend to settle in the end. This allows the user to pick the middle value that is not influenced by motion artifacts.

These computed median reference values of peak-to-peak & peak-to-valley are then used as features for the HbA1c algorithm to predict a real-time HbA1c range of the user. ECG module 716 In operation, For ECG measurement, the ECG module 716 is configured to perform receiving the denoised signal as the matrix of signal (n×7); receiving input from set of Dry electrodes which acts as one of the sensors for ECG module, perform motion artifact removal from the denoised signal; processing the motion artifact removed signal to identify the heart rate with multiple frequency filters, wherein the heart rate is present only in the frequency range of 0.663 to 3.66 Hz; and passing the filtered signals through a series of thresholding functions, wherein each peak that is observed after the set threshold functions is considered as a heart beat, and a number of peaks detected in the signal in a defined time frame with respect to their timestamp are utilized to generate an ECG report to understand sinus rhythm and other parameters of the heart.

Hence, motion artifact removed signals are fed into the ECG computation algorithm where the algorithm identifies the heart rate with multiple frequency filters, heart rate is present only in the frequency range of 0.663 to 3.66 Hz this particular frequency is filtered & later passes through series of thresholding functions and each peak that is observed after the set threshold functions can be considered as a heart beat. Number of peaks noted in signal that is captured in a known time frame helps along with the timestamp at which the peak is noted, here a series of timestamps are noted at each respective peak in the array and the normal is computed to know the Average time interval for each peak which can help us understand the Sinus Rhythm and other parameters of the heart. Heart Rate Module 718, In operation, for heart rate measurement, the heart rate module 718 is configured to perform receiving the denoised signal as the matrix of signal (n×7); perform motion artifact removal from the denoised signal; processing the motion artifact removed signal to identify the heart rate with multiple frequency filters, wherein the heart rate is present only in the frequency range of 0.663 to 3.66 Hz; passing the filtered signal through a series of thresholding functions, wherein each peak that is observed after the set threshold functions is considered as a heart beat, and a number of peaks detected in the signal in a defined time frame with respect to their timestamp are utilized to measure the heart rate of the user.

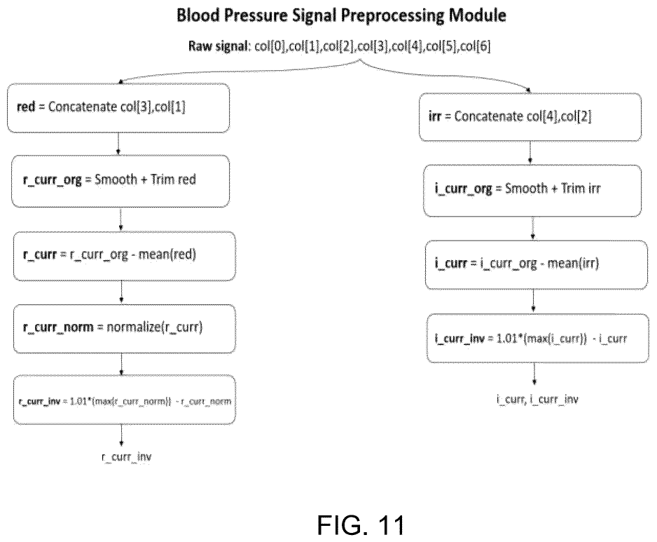

In brief, a motion artifact removed signal is fed into the heart rate algorithm where the algorithm identifies the heart rate with multiple frequency filters, heart rate is present only in the frequency range of 0.663 to 3.66 Hz. This particular frequency is filtered & later passes through a series of thresholding functions and each peak that is observed after the set threshold functions can be considered a heartbeat. A number of peaks noted in a signal that is captured in a known time frame help to calculate beats per minute, i.e., heart rate, challenges in heart rate measurement can be detecting the motion artifact as a heart beat and using them in the computation of heart rate calculation. Blood Pressure Module 720, In operation, as shown in FIG. 11, for blood pressure measurement, the blood pressure module 720 is configured to perform separating the denoised signal obtained as a matrix of signals (n×7) into two component signals: red component and irr component, wherein the red component is formed by concatenating zero-indexed columns 3 and 1, which denotes the signal that is obtained from the reflected light of red signal of wavelength 660 nm, and wherein the irr component is formed by concatenating the zero-indexed columns 4 and 2, which denotes the signal that is obtained from the reflected IR light of wavelength 880 nm; ultra lowpass conditioning and trimming both the components; subtracting both the ultra lowpass conditioned signal and trimmed components (r curr, i curr) by a normal of the respective component signals, for the red component (r curr), normalizing it and computing its inverse representation (r curr inv) by multiplying 1.01 by a maximum value in this normalized signal and then subtracting each element of the normalized signal from this value; for the irr component (i curr), multiply 1.01 by a maximum value in the irr component and then subtract each element of the signal from this value to obtain i curr inv. finding indices of peaks in the signals i curr inv and i curr as maxindices and minIndices; implementing the maxindices to index into r curr inv to get an array of values (r curr inv maxima); implementing minIndices to index into r curr inv to get another array of values (r curr inv minima); converting both of these arrays to the same length by only considering values up until the length of the smaller of the two arrays; and once both of these arrays are obtained as two arrays of equal length, computing their respective normals, add both their normals up and divide this sum by 2 to obtain the input to the equations for computing the systolic and diastolic blood pressure values, wherein the equations for both systolic and diastolic blood pressure are Polynomial equations, whose coefficients are determined by a pre-training a regression classifier of the AI-based model.

In brief, in order to calculate blood pressure, first, the raw signal is to be separated into two component signals: red component and irr component. The red component is formed by concatenating zero-indexed columns 3 and 1, which denotes the signal that we get from the reflected light of Red led of 660 nm & the irr component is formed by concatenating the zero-indexed columns 2 and 4 which denotes the signal that we get from the reflected light of NIR led of wavelength 880 nm Then, both components are ultra lowpass conditioned, trimmed and each element is subtracted by the normal of the respective component signals. A call is made for each component obtained thus far, r curr and i curr. After this step, for the red component (r curr), normalize it and compute its inverse representation (r curr inv) by multiplying 1.01 by the maximum value in this normalized signal and then subtracting each element of the normalized signal from this value. On the other hand, for the irr component (i curr), multiply 1.01 by the maximum value in the irr component and then subtract each element of the signal from this value to obtain i curr inv.

Blood oxygen level (SPo2) Module 722 For blood oxygen level (SPo2) measurement, the blood oxygen level (SPo2) module 722 is configured to perform receiving the PPG signal at the photodetector; measuring saturation of blood by oxygen from PPG signal received at two wavelengths including red signals received from LED and IR signals received from NIR; calculating the blood oxygen (SpO2) level is calculated from a calibration R curve plotting SpO2 versus ratio red and IR signals, wherein the R is calculated as R=(Rac/Rdc)/(IRac/IRdc) where Rac is the pulsating AC component of the fingertip by red signal and Rdc is the non-pulsating DC component of the fingertip by a red signal of wavelength 660 nm, and IRac and IRdc are also pulsating AA and DC components of the fingertip by the IR signal of wavelength 880 nm.

Body temperature Module For measurement of the body temperature, the temperature sensor is configured to measure the temperature of the user based on the IR signal of wavelength 880 nm obtained from the NIRs. Motion Artefact Removal, Motion Artifacts can be a major noise contributor to the PPG signal and it becomes excruciatingly difficult to predict vitals like Glucose without a legitimate process to remove motion artifacts. Motion Artefact apperception and filtering is an adamantine process due to the nature of motion artifact lying in the same frequency where other key features of vitals are observed, It's easy to identify motion artefacts through a visual inspection and trail to run it through a band-pass or low pass filters which can show a compelling result of removing the artefacts but a reduction of quality features for measurement of key vitals is observed, a heterogeneous level of DWT and different levels of thresholding can give a decent result of removal of motion artifact while maintaining the integrity of the features in the signal Removing the motion artifact components from the signal directly may not yield accurate results as filtering them out, sometimes, can cause feature loss. For better accuracy of the measurements, the present invention includes a finger pressure analysis method wherein, the present described computation models compute the vitals first and later adjust the measurements based on the pressure exerted by the user. Pressure Analysis is done during the computation of vitals, where the pressure is identified and removed. This can be done by using various modules like ultra low pass filter regression models and AI. The computation modules compute the vitals first, later based on the pressure exerted by the user the delta difference is adjusted from the vitals. The delta differences are calculated by a regression model which understands pressure components and can provide the necessary delta correction. This approach showed compelling results than removing the noise components directly from the signal.

Ultra low pass Filter is a key process that helps to obtain a fair signal out of the raw signal that we get from the hardware unit is removing a baseline drift from the raw signal the baseline drift is observed by factors like user respiration rate and sometimes motion artefacts effect the baseline drift that the signal consists of, this baseline drift can be identified by calculating the Ultra low pass filter for the entire signal. Ultra Lowpass Conditioned Signa, the Ultra lowpass conditioned Signal is subtracted from the raw signal resulting in a signal where baseline drift is removed. A Signal that has baseline drift removed is helpful for calculations like heart rate and ECG where the peaks are better identified than a Signal that has a baseline drift.

Ultra lowpass conditioned Signal can be helpful in many ways and we can take the advantage of the custom conditioning to efficiently eliminate artefacts like a baseline drift and information about respiration rate based on the signal conditioning. Similarly, the other advantages of the ultra Lowpass conditioned signal are discussed in the segment of the key features.

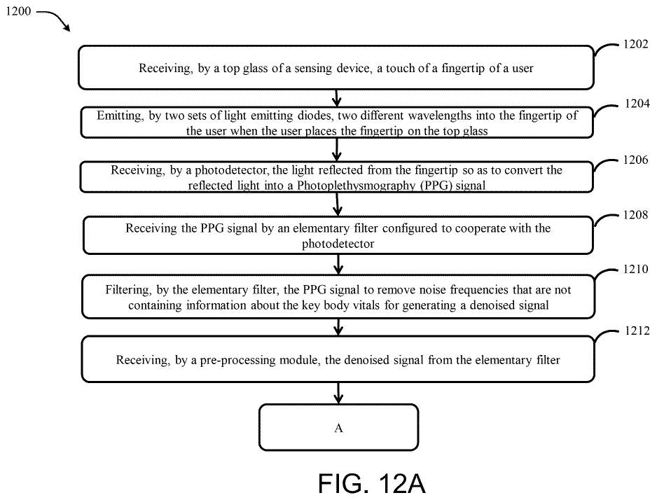

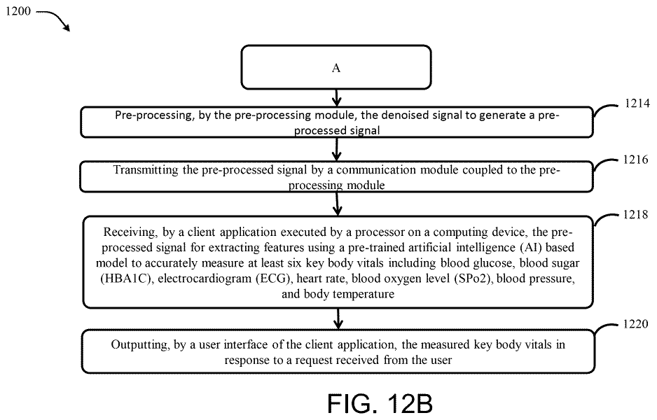

FIG. 12 refers to an example method 1200 for operating a non-invasive sensing system for measuring key body vitals. The order in which method 1200 is described is not intended to be construed as a limitation, and any number of the described method blocks may be combined in any order to implement method 1200, or an alternative method. Furthermore, method 1200 may be implemented by processing resource or computing device(s) through any suitable hardware, non-transitory machine-readable medium/instructions, or combination thereof. At block 1202, method 1200 includes receiving, by a top glass of a sensing device 102, a touch of a fingertip of a user. At block 1204, method 1200 includes emitting, by a light emitting unit mounted underneath the top glass with two sets of light emitting diodes 202-1, 202-2, two different wavelengths into the fingertip of the user when the user places the fingertip on the top glass. At block 1206, method 1200 includes receiving, by a photodetector 204 mounted underneath the top glass, the light reflected from the fingertip so as to convert the reflected light into a Photoplethysmography (PPG) signal. At block 1208, the method 1200 includes receiving the PPG signal by an elementary filter 206 configured to cooperate with the photodetector 204.

At block 1210, the method 1200 includes filtering, by the elementary filter 204, the PPG signal to remove noise frequencies that are not containing information about the key body vitals for generating a denoised signal. At block 1212, the method 1200 includes receiving, by a pre-processing module 208, the denoised signal from the elementary filter 206, wherein the denoised signal is obtained as a matrix of signals (n×7), and wherein said matrix of signals includes 3000 data samples with a sampling frequency of 100 samples per second and a sampling period of 30 seconds. At block 1214, the method 1200 includes pre-processing, by the preprocessing module 208, the denoised signal to generate a pre-processed signal. At block 1216, the method 1200 includes transmitting the pre-processed signal by a communication module 210 coupled to the pre-processing module 208. At block 1218, the method 1200 includes receiving, by a client application 708 executed by a processor 702 on a computing device 106, the pre-processed signal for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature. At block 1220, the method 1200 outputs, by a user interface 706 of the client application 708, the measured key body vitals in response to a request received from the user.

As per another embodiment of the present invention, badging features of the user for example, demographic information can be analysed. Based on the n×7 matrix received from the device, a badging analysis is performed on the signal by studying multiple components of the signal to understand various badging features of the user including demographic information. This badged signal can help with an in-depth analysis of various vital computations. A pre-trained AI model is used for the segmentation analysis.

Ultralow pass conditioned signal contains fair information that is not just baseline drift, it is observed that Ultralow pass conditioned signal shows a downward trend when the sample collection of the user happened while he/she was fasting or when their glucose trend was also going down like a postprandial state of 2 hours and observed that the Ultralow pass conditioned signal shows a fairly stable trend when even the glucose levels are stable. The Ultralow pass conditioned signal contains information about respiration which helps us to calculate the respiration rate. Multi-level DWT can yield de-noised signals which contain key features for the classification of vitals like Glucose, but heavy processing is required for consistent & accurate results. Classification of Diabetic and Non-Diabetic using real-time PPG signals is possible by Multi-level DWT and Pre-processing filters like Chebyshev type 2 order 4 filters. The 1st and 2nd order Derivative of raw signal with respect to time does contain features for the classification of BGL levels.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A non-invasive sensing system for measuring key body vitals, said system comprising:
a sensing device comprising a top glass configured to receive a touch of a fingertip of a user, wherein the sensing device includes:
a light emitting unit mounted underneath the top glass, said light emitting unit comprising two sets of light emitting diodes to emit two different wavelengths into the fingertip of the user when the user places the fingertip on the top glass;
a photodetector, mounted underneath the top glass, to receive the light reflected from the fingertip and convert the light into a Photoplethysmography (PPG) signal;
an elementary filter configured to cooperate with the photodetector to receive the PPG signal, filter the PPG signal to remove noise frequencies that are not containing information about the key body vitals, and generate a denoised signal;
a pre-processing module to receive the denoised signal from the elementary filter, wherein the denoised signal is obtained as a matrix of signals (n×7), wherein said matrix of signals includes 3000 data samples with a sampling frequency of 100 samples per second and a sampling period of 30 seconds, and wherein said pre-processing module is configured to pre-process the denoised signal to generate a pre-processed signal; and a communication module coupled to the pre-processing module to transmit the pre-processed signal;
a client application, executed by a processor on a computing device, to receive the pre-processed signal for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature; and
a user interface, of the client application, to receive a request for the measured key body vitals and to output the measured key body vitals.

2. The non-invasive sensing system of claim 1, wherein the pre-processing module receives the matrix of signal (n×7) to perform:
concatenation of two parts consisting in the matrix of signals (n×7) including 3000 data samples to generate a concatenated signal, where a first part of the matrix of signals (n×7) lies in 3rd column of said matrix and a second part of the matrix of signals (n×7) lies 1st column of said matrix;
trimming of the concatenated signal to discard a part from the beginning of capturing the PPG signal and a part from the ending of capturing the PPG signal so as to generate a trimmed signal, wherein the part from the beginning includes about 300 data samples and the part from the ending includes about 100 data samples from the array of 3000 data samples;
performing Ultra Low pass conditioning on the trimmed signal for extracting the key component of the trimmed signal to generate a smooth signal; and
generating the pre-processed signal as a pre-processed difference signal computed based on a difference between the Ultralow pass conditioned signal and the trimmed signal.

3. The non-invasive sensing system of claim 1, wherein for blood glucose measurement, the client application further comprises a blood glucose module to receive the Ultralow pass conditioned signal from the pre-processing module of the sensing device, wherein the blood glucose module comprises:
a glucose conditioning module to receive the Ultralow pass conditioned signal from the pre-processing module of the sensing device and compute a normal value of the key two dimensional components analyzed by AI and a conditioning interference module to receive the computed Normal Value as a parameter, wherein the conditioning module is configured to compute a user's blood glucose value based on a range in which the computed Normal value falls, wherein for computing the user's blood glucose value, the conditioning interference module uses a polynomial equation where an input is the computed normal value and coefficients for each range are determined by pre-training a regression classifier of the AI-based model.

4. The non-invasive sensing system of claim 2, wherein for blood sugar measurement, the client application further comprises a blood sugar module (HbA1c) to receive the pre-processed difference signal from the pre-processing module of the sensing device, wherein the blood sugar module (HbA1c) is configured to perform:
batching the pre-processed difference signal into an M number of batches of an N number of samples, where N=50; for each batch, simultaneously computing a peak-to-peak value and an average peak-to-valley for each peak in the batch, computing median reference values of the peak-to-peak and the peak-to-valley values of entire batches of the key components of overall length of the difference signal analyzed by the AI categorization, implementing the pre-trained AI-based model to receive the median reference values as the extracted features for predicting the blood sugar (HbA1C).

5. The non-invasive sensing system of claim 1, wherein for ECG measurement, the client application further comprises an ECG module to perform:
receiving the denoised signal as the matrix of signal (n×7); perform motion artefact removal from the denoised signal; processing the motion artefact removed signal to identify the heart rate with multiple frequency filters, wherein the heart rate is present only in the frequency range of 0.663 to 3.66 Hz; and passing the filtered signal through a series of thresholding functions, wherein each peak that is observed after the set threshold functions is considered as a heart beat, and a number of peaks detected in the signal in a defined time frame with respect to their timestamp are utilized to generate an ECG report to understand sinus rhythm of the heart and other components of the heart

6. The non-invasive sensing system of claim 1, wherein for heart rate measurement, the client application further comprises a heart rate module to perform:
receiving the denoised signal as the matrix of signal (n×7); perform motion artefact removal from the denoised signal; processing the motion artefact removed signal to identify the heart rate with multiple frequency filters, wherein the heart rate is present only in the frequency range of 0.663 to 3.66 Hz;
passing the filtered signal through a series of thresholding functions, wherein each peak that is observed after the set threshold functions is considered as a heart beat, and a number of peaks detected in the signal in a defined time with respect to their timestamp are utilized to measure the heart rate of the user.

7. The non-invasive sensing system of claim 1, wherein said two sets of light emitting diodes includes:
Light Emitting Diodes (LED) with a wavelength of 660 nm; and
Near Infra-Red LED (NIR) with a wavelength of 880 nm.

8. The non-invasive sensing system of claim 1, wherein the photodetector is configured with spectral range sensitivity of 600 to 5000 nm.

9. The non-invasive sensing system of claim 7, wherein for blood oxygen level (SPo2) measurement, the client application further comprises a blood oxygen level (SPo2) module to perform:
receiving the PPG signal at the photodetector;
measuring saturation of blood by oxygen from PPG signal received at two wavelengths including red signals received from LED and IR signals received from NIR;
calculating the blood oxygen (SpO2) level is calculated from a calibration R curve plotting SpO2 versus ratio red and IR signals, wherein the R is calculated as:
R=(Rac/Rdc)/(IRac/IRdc), where Rac is the pulsating AC component of the fingertip by red signal and Rdc is the non-pulsating DC component of the fingertip by a red signal of wavelength 660 nm, and IRac and IRdc are also pulsating AA and DC components of the fingertip by the IR signal of wavelength 880 nm.

10. The non-invasive sensing system of claim 7, wherein for blood pressure measurement, the client application further comprises a blood pressure module to perform:
separating the denoised signal obtained as a matrix of signals (n×7) into two component signals: red component and irr component, wherein the red component is formed by concatenating zero-indexed columns 3 and 1, which denotes the signal that is obtained from the reflected light of red signal of wavelength 660 nm, and wherein the irr component is formed by concatenating the zero-indexed columns 4 and 2, which denotes the signal that is obtained from the reflected IR light of wavelength 880 nm;
Ultra lowpass conditioning and trimming both the components; subtracting both the Ultra lowpass conditioned signal and trimmed components (r curr, i curr) by the normal of the respective component signals,
for the red component (r curr), normalising it and computing its inverse representation (r curr inv) by multiplying 1.01 by a maximum value in this normalised signal and then subtracting each element of the normalised signal from this value;
for the irr component (i curr), multiply 1.01 by a maximum value in the irr component and then subtract each element of the signal from this value to obtain i curr inv. finding indices of peaks in the signals i curr inv and i curr as maxindices and minIndices;
implementing the maxindices to index into r curr inv to get an array of values (r curr inv maxima);
implementing minIndices to index into r curr inv to get another array of values (r curr inv minima);
converting both of these arrays to the same length by only considering values up until the length of the smaller of the two arrays; and once both of these arrays are obtained as two arrays of equal length, computing their respective normals, add both their normals up and divide this sum by 2 to obtain the input to the equations for computing the systolic and diastolic blood pressure values, wherein the equations for both systolic and diastolic blood pressure are Polynomial equations, whose coefficients are determined by a pretraining a regression classifier of the AI-based model.

11. The non-invasive sensing system of claim 1, wherein the elementary filter is a Finite Impulse Response (FIR) band-pass filter with band frequencies from 0.5 Hz to 5.0 Hz and gain of 1100, and wherein the FIR band-pass filter is configured to remove the noise frequencies lying beyond a frequency range of 0.5 Hz to 5 Hz.

12. The non-invasive sensing system of claim 1, wherein the top glass allows wavelength frequencies of 660 nm to 880 nm with about 85%-95% transmission percentage.

13. The non-invasive sensing system of claim 1, wherein the sensing device comprises a black optical sensor shield that covers the boundaries of the light emitting unit and the photodetector for absorbing unwanted light that might bounce back to the photodetector.

14. The non-invasive sensing system of claim 1, wherein the sensing system further measures the differential analysis of blood by utilizing more than one finger of the user, at a time, at the time of data collection.

15. The non-invasive sensing system of claim 1, wherein the sensing system implements finger pressure analysis wherein, the computation models compute the vitals first and later adjust the key vital measurements based on the pressure exerted by the user.

16. A method for operating a non-invasive sensing system for measuring key body vitals, said method comprising:
receiving, by the top glass of a sensing device, a touch of a fingertip of a user;
emitting, by a light emitting unit mounted underneath the top glass with two sets of light emitting diodes, two different wavelengths into the fingertip of the user when the user places the fingertip on the top glass; receiving, by a photodetector mounted underneath the top glass, the light reflected from the fingertip so as to convert the reflected light into a Photoplethysmography (PPG) signal;
receiving the PPG signal by an elementary filter configured to cooperate with the photodetector;
filtering, by the elementary filter, the PPG signal to remove noise frequencies that are not containing information about the key body vitals for generating a denoised signal;
receiving, by a pre-processing module, the denoised signal from the elementary filter, wherein the denoised signal is obtained as a matrix of signals (n×7), wherein said matrix of signals includes 3000 data samples with a sampling frequency of 100 samples per second and a sampling period of 30 seconds;
pre-processing, by the pre-processing module, the denoised signal to generate a pre-processed signal;
transmitting the pre-processed signal by a communication module coupled to the pre-processing module;
receiving, by a client application executed by a processor on a computing device, the pre-processed signal for extracting features using a pre-trained artificial intelligence (AI) based model to accurately measure at least six key body vitals including blood glucose, blood sugar (HBA1C), electrocardiogram (ECG), heart rate, blood oxygen level (SPo2), blood pressure, and body temperature; and
outputting, by a user interface of the client application, the measured key body vitals in response to a request received from the user.
